(19) United States
(12) Patent Application Publication
(10) Pub. No.: US 2025/0064942 A1
(43) Pub. Date: Feb. 27, 2025

(54) ARTIFICIAL OXYGEN CARRIER BLOOD PRODUCT

(71) Applicant: Andre Danesh, Brookline, MA (US)

(72) Inventor: Andre Danesh, Brookline, MA (US)

(21) Appl. No.: 18/642,932

(22) Filed: Apr. 23, 2024

Related U.S. Application Data
(63) Continuation-in-part of application No. 18/422,426, filed on Jan. 25, 2024.
Application No. 18/422,426 is a continuation-in-part of application No. 18/455,115, filed on Aug. 24, 2023.

(51) Int. Cl. A61K 47/54; A61K 9/00; A61K 45/06

(52) U.S. Cl. CPC A61K 47/547; A61K 9/0026; A61K 45/06

(57) ABSTRACT

The invention comprises a process and composition relating to the use of an artificial blood that is a pharmaceutically acceptable solution of an iron amino acid chelate. The composition is directed to a hemoglobin free artificial blood composition made from one or more amino acids known to be bound to the hemoglobin molecule but free of hemoglobin for purposes of this invention. The hemoglobin free amino acids are chelated with iron (ferrous or ferric), oxygenated, and used as an artificial blood replacement for whole blood or as a component of an artificial blood composition. The iron component of the amino acid chelate is oxygenated to enable the chelate to carry oxygen throughout the body. To form a solution suitable for transfusion, the oxygen bearing iron chelate of the amino acid is dissolved, to the extent necessary, preferably in an isotonic saline solution. In use, the hemoglobin free artificial blood composition is transfused into an individual suffering trauma due to loss of blood using conventional transfusion procedures know in the art.

### CROSS-REFERENCE TO RELATED PATENT APPLICATIONS

This application is a continuation-in-part of co-pending U.S. patent application Ser. No. 18/422,426, filed Jan. 25, 2024, which in turn is a continuation-in-part of co-pending U.S. patent application Ser. No. 18/445,115, filed Apr. 17, 2023.

### FIELD OF THE INVENTION

The invention relates generally to the development of artificial blood compositions. More specifically, the invention relates to pharmaceutically acceptable hemoglobin free oxygenated iron amino acid chelates for use in the formulation of an artificial blood product and methods for use of the same.

### BACKGROUND OF THE INVENTION

Circumstances such as accidents, surgery, traumatic hemorrhagic shock, and other casualties cause major blood loss. Allogenic blood transfusion can be resuscitative for such conditions; however, it has numerous ambivalent effects, including supply shortage, needs for more time, cost for blood grouping, the possibility of spreading an infection, and short shelf-life. Hypoxia or ischemia causes heart failure, neurological problems, and organ damage in many patients. To address this emergent medical need for resuscitation and to treat hypoxic conditions as well as to enhance oxygen transportation, researchers aspire to achieve a robust technology with an object of developing safe and feasible blood substitutes, or artificial blood, for effective oxygen transport.

Efforts to produce artificial blood were spurred on by the military in World Wars I and II and, more recently, by the discovery in the early 1980s that HIV could be transmitted by blood transfusion. Blood is now safe, thanks to improved collection and screening by blood banks. But it still has to be cross-matched and can be stored for only a few weeks before it has to be discarded. If a solution that could replace blood were immediately available, if it were completely safe, and if it could be stored for long periods; it would be extremely useful in emergencies, disaster and wars—not to mention in countries where blood is not collected and stored as it is in the U.S and Europe.

Artificial blood is designed to transport oxygen and carbon dioxide through the body. No substitutes have yet been invented that can replace the other vital functions of blood: coagulation and immune defense. Therefore, the replacement solutions being developed today are more accurately described as oxygen carriers. There are basically two types of oxygen carriers which differ in the way in which they transport oxygen. One is based on perfluoro chemicals, the other on hemoglobin.

The following discussion relates to hemoglobin blood substitutes. Hemoglobin-based oxygen carriers (HBOCs) utilize the same oxygen-carrying protein molecule found in blood where the oxygen bonds chemically to hemoglobin. HBOCs differ from red blood cells in that the hemoglobin is not contained within a membrane. The membrane of a red blood cell contains the antigen molecules that determine the ‘type’ of the blood (A, B, AB or O). In addition, these artificial oxygen carriers can be stored for long periods of time greatly simplifying the work of the blood bank. Best of all, HBOCs can be used in situations and locations where real blood is not available, as at disaster sites, underdeveloped countries or battle zones. Moreover, unlike red blood cells, blood substitutes can be pasteurized, filtered and chemical-cleansed to make them sterile. These procedures remove microorganisms responsible for diseases such as AIDS and hepatitis. Because the substitutes do not have cell membranes with blood-group antigens, cross-matching and typing are not required before use. This saves time and facilities and allows on-the-spot transfusion. Furthermore, blood substitutes can be stored for more than one year, as compared with about one month for donor blood stored using standard methods.

Two problems arise when hemoglobin is removed from the red blood cells and these problems account for the large amount of scientific research that has been conducted so far in this area. First, the red cell membrane protects hemoglobin from degradation and protects tissues from the toxic effects of free hemoglobin. Second, when oxygen is delivered by a cell-free carrier instead of red blood cells, complex biological mechanisms alter the flow through the smallest blood vessels (the arterioles and capillaries). Major advances have been made in overcoming both of these problems, and several HBOC products are now in advanced human trials. It is anticipated that within the next several years, the first of these products will become available to physicians for use in patients.

Hemoglobin is the protein in red blood cells that is responsible for carrying oxygen from the lung to the other tissues. Therefore, current methods to formulate blood substitutes are to use hemoglobin extracted from red blood cells. However, raw hemoglobin extracted from red blood cells cannot be used as a blood substitute. Each hemoglobin molecule is a tetramer that consists of four subunits. When infused into the body, a hemoglobin molecule breaks down into potentially toxic half molecules, or dimers. There are also other problems related to hemoglobin in free solution. The challenge to those engaged in research based upon hemoglobin is to modify the hemoglobin to allow it for use as a blood substitute.

First generation hemoglobin blood substitutes rely on molecular modifications of hemoglobin, either by chemically cross-linking the molecules or by modifying them using recombinant DNA technology. So-called bifunctional agents can cross-link the hemoglobin molecules to one another to form polyhemoglobin. The basic idea of cross-linked hemoglobin and encapsulated hemoglobin dates back to the 1960s (T. M. S. Chang in Science, Vol. 146, page 524; 1964, and H. F. Bunn and J. H. Jandl in the Transactions of the Association of American Physicians, Vol. 81, page 147; 1968). The cross-linked hemoglobin molecules are stable and do not break down. Some bifunctional agents can also cross-link each hemoglobin molecule internally to prevent its breakdown into dimers. Recombinant technology applied to the bacterium E. coli can produce altered hemoglobin molecules that do not break down into half molecules. Hemoglobin can also be cross-linked to soluble polymers to form so-called conjugated hemoglobin. All the above modifications also result in blood substitutes that have a greater ability to release oxygen to the tissues than do red blood cells. Clinical trials in humans are ongoing using products from a number of companies. In the case of polyhemoglobin, Northfield is now in Phase III (large-scale efficacy) clinical trials that infuse up to 5,000 milliliters of blood substitutes into surgical patients. The company is using pyridoxalated glutaraldehyde cross-linked human hemoglobin. Biopure is in Phase II (small-scale efficacy) clinical trials using pyridoxalated glutaraldehyde cross-linked bovine hemoglobin. Hemosol is in Phase II clinical trials in surgical patients, using a new cross-linker to form a molecule known as o-raffinose cross-linked human polyhemoglobin. In the case of intra-molecularly cross-linked hemoglobin, Baxter is now in Phase III clinical trials with a large number of surgical patients; the company is using Diaspirin cross-linked human hemoglobin. Somatogen is now deep into their Phase II clinical trials with their recombinant human hemoglobin. For conjugated hemoglobin, Enzon is now in Phase II clinical trials, and Apex is now in Phase I (safety) clinical trials.

Further discussion of artificial blood may be found in the following publications: Hemoglobin-Based Red Cell Substitutes. Robert M. Winslow. Johns Hopkins University Press, 1992; Blood Substitutes—A Moving Target. Robert M. Winslow in Nature Medicine, Vol. 1, No. 11, pages 1212-1215; 1995; Blood Substitutes. Robert M. Winslow in Science & Medicine, Vol. 4, No. 2, pages 54-63; 1996; Artificial Oxygen Carriers and Red Blood Cell Substitutes: An Historic Overview and Recent Developments Toward Military and Clinical Relevance: Christopher Bialas, Christopher Moser, C. Sims, Medicine The Journal of Trauma and Acute Care Surgery, 2019; From hemoglobin allostery to hemoglobin-based oxygen carriers, S. Faggiano, L. Ronda, A. Mozzarelli, Biology, Medicine, Molecular aspects of medicine, 2021.

Though efforts to develop an artificial blood based upon hemoglobin are in clinical trials, the required hemoglobin needed for the formulation of an artificial blood product relies upon using either native or recombinant human hemoglobin, modified forms of human hemoglobin or modified forms of hemoglobin from other species. Unmodified hemoglobin can be used as an oxygen therapeutic; however, it can bind NOx and causes severe vasoconstriction and hypertension. As a consequence of its molecular weight, hemoglobin can cause significant toxicities, especially to the kidney where it clogs the glomerular apparatus. As a consequence, the majority of tested hemoglobin in humans are modified to prolong their half-life and reduce their toxicity. Consequently, hemoglobin as an artificial blood is costly and difficult to acquire and store.

### SUMMARY OF THE INVENTION

It is known that a hemoglobin molecule is made up of four polypeptide chains, two alpha chains of 141 amino acid residues each and two beta chains of 146 amino acid residues each. The subject invention is directed to a hemoglobin free artificial blood composition made from one or more essential and/or non-essential amino acids known to be bound to the hemoglobin molecule. The chelates formed by hemoglobin free amino acids chelated with iron [ferrous or ferric], oxygenated, and used as an artificial blood replacement for whole blood or as a component of an artificial blood composition. The iron component of the amino acid chelate is oxygenated to enable the chelate to carry oxygen throughout the body. Oxygenation is accomplished using methods known to the art and employed to oxygenate hemoglobin. One method involves passing a stream of oxygen through a solution of the amino acid chelate or contacting the amino acid solution with hydrogen peroxide using procedures known in the art and conventionally used for the oxidation of hemoglobin. To form a solution suitable for transfusion, the oxygen bearing iron chelate of the amino acid is dissolved, to the extent necessary, in a suitable solution such as an isotonic saline solution. In use, the hemoglobin free artificial blood composition is transfused into an individual suffering trauma due to loss of blood using conventional transfusion procedures know to the art.

### DESCRIPTION OF THE PREFERRED EMBODIMENTS

Non-hemoglobin artificial blood derived from amino acid chelates is believed to represent a promising avenue in the quest for blood substitutes. Unlike traditional hemoglobin-based solutions, which often face challenges such as toxicity and limited oxygen-carrying capacity, this innovative approach harnesses the unique properties of iron amino acid chelates to mimic the oxygen transport function of natural blood.

Amino acid chelates are compounds wherein ferrous or ferric ions are bound to amino acids to form stable complexes. These chelates are expected to exhibit excellent biocompatibility and can be engineered to replicate the oxygen delivery capability of hemoglobin. This has the potential to address critical issues associated with traditional blood substitutes, such as vasoconstriction and poor tissue oxygenation.

A significant advantage of hemoglobin free artificial blood is its reduced risk of toxicity. Hemoglobin-based solutions often struggle with side effects, including oxidative stress and vasoconstriction, which can lead to adverse physiological reactions. Amino acid chelates, being biologically compatible, offer a safer alternative, minimizing the risk of unwanted effects and improving the overall biocompatibility of artificial blood.

In addition, the versatility of amino acids allows for precise control over the properties of the artificial blood. By manipulating the composition of iron based amino acid chelates, it is possible to tailor the oxygen affinity, release kinetics, and stability of the synthetic blood substitute. This level of customization is a significant advantage in designing a blood substitute that closely mimics the characteristics of natural blood.

Another noteworthy advantage is the potential for sustained oxygen release. Iron amino acid chelates can be engineered to release oxygen gradually thereby preventing abrupt fluctuations that may occur with other blood substitutes. This controlled release is crucial for ensuring optimal oxygen delivery to tissues, reducing the risk of oxygen toxicity, and improving the overall efficacy of the artificial blood.

A further advantage artificial blood derived from amino acid chelates is the availability of such chelates as a consequence of substantial research devoted to development of these materials for use as nutritional supplements. Consequently, they are readily available and low in cost whereby an artificial blood product derived from these materials would be significantly lower in cost than a blood substitute derived from hemoglobin. Moreover, due to the ready availability of the iron amino chelates, a hemoglobin free blood substitute may be formulated from available iron amino acid chelates with reliance on hemoglobin as a source of the chelates.

Metal amino acid chelating complexes, inclusive of ferrous and ferric iron complexes, are well known to the art as they have found extensive applications in various fields of human interest. Amino acids are the building blocks of proteins, which are vital for many bodily functions. When a person eats a food that contains a protein, that person's digestive system breaks the protein down into amino acids. There are numerous essential and non-essential amino acids acquired from food or supplements. The body combines the amino acids in various ways to carry out bodily functions. A healthy body can manufacture needed amino acids, so these amino acids do not usually need to enter the body through the diet. These amino acids build muscles, enable chemical reactions in the body, transport nutrients, prevent illness, and carry out other functions. Amino acid deficiency can result in decreased immunity, digestive problems, depression, fertility issues, lower mental alertness, slowed growth in children, and many other health issues. To treat amino acid deficiency, amino acids are used as supplements and these supplements are often administered in the form of an iron amino acid chelate.

As a consequence of the extensive use of amino acid chelates as supplements, methods for the synthesis of such chelates are well known and disclosed in numerous publications known to the art. Consequently, procedures for the synthesis of iron chelates of an amino acid is not considered to be a part of the invention disclosed herein. The structure, chemistry and the bioavailability of amino acid chelates are described in numerous documents, for example, Ashmead et al, U.S. Pat. No. 4,020,158 granted Aug. 26, 1977; Ashmead, U.S. Pat. No. 4,167,564 granted Sep. 11, 1979; Jensen, U.S. Pat. No. 4,216,143 granted Sep. 11, 1979; Mayo, U.S. Pat. No. 4,721,644 granted Jan. 26, 1988; Ashmead, U.S. Pat. No. 4,599,152 granted Jul. 8, 1986; Ashmead, U.S. Pat. No. 4,774,089 granted Sep. 27, 1988; Ashmead, U.S. Pat. No. 4,830,716 granted May 6, 1989; Ashmead et al, U.S. Pat. No. 4,863,898 granted May 9, 1989; Ashmead et al, U.S. Pat. No. 4,725,427 granted Feb. 16, 1988; Ashmead et al., Chelated Mineral Nutrition, (1982), Chas. C. Thomas Publishers, Springfield, III, especially Chapter 2.; Ashmead et al., Intestinal Absorption of Metal Ions, C. Thomas Publishers, Springfield (1985); Ashmead et al., Foliar Feeding of Plants with Amino Acid Chelates, (1986); and Ashmead, Amino Acid Chelation in Human and Animal Nutrition, CRC Press, 2012; each incorporated herein by reference for their teaching of the synthesis of metal amino acid chelates and their uses.

As is known in the art, amino acid chelates are generally made by reacting alpha-amino acids and metal ions, where the metal ion has a valence of 2 or more, to form a ring structure in the form of a chelate. In such a reaction, the cationic charge of the metal ion is neutralized by the free amino group or carboxyl group of the alpha-amino acid.

One such representative metallic amino acid chelate and representative reaction to form this chelate is represented by the formula below:

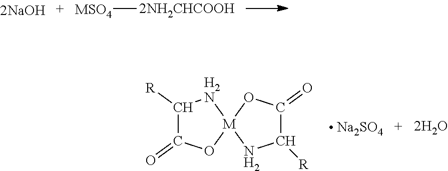

where M is a divalent metal ion, and R is a side chain of naturally occurring amino acids or peptides. The metal ion M contemplated herein is iron having a oxidation state of 2 or 3.

As stated above, the invention disclosed herein is a hemoglobin free oxygenated ferrous or ferric chelate of an amino used to form an artificial blood composition where the amino acid[s] is one or more of those amino acids known to be bonded to the hemoglobin molecule. In other words, the artificial blood composition is a liquid comprising one or more amino acids known to be bonded to hemoglobin, but for purposes of this invention, free of hemoglobin.

The amino acids contemplated for use as an artificial blood are known essential and non-essential amino acids. The following Table 1 and the examples that follow Table 1 depict those amino acids known to be bonded to the hemoglobin molecule. The specific amino acid contemplated is shown in column 1 and column 2 provides the number of each of the amino acid chains known to be bonded to the hemoglobin molecule. Table 1 discloses the most preferred essential amino acids used to formulate an artificial blood composition in accordance with this invention while the amino acids following Table 1 are contemplated by the invention, but are lesser preferred.

TABLE 1

| Amino Acid | Alpha Chain | Beta Chain |
|---|---|---|
| Alanine (Ala) | 21 | 15 |
| Arginine (Arg) | 3 | 3 |
| Asparagine (Asn) | 4 | 6 |
| Aspartic Acid (Asp) | 8 | 7 |
| Cysteine (Cys) | 1 | 2 |
| Glutamine (Gln) | 1 | 3 |
| Glutamic Acid (Glu) | 4 | 8 |
| Glycine (Gly) | 7 | 13 |
| Histidine (His) | 10 | 9 |
| Isoleucine (Ile) | 0 | 0 |
| Leucine (Leu) | 18 | 18 |
| Lysine (Lys) | 11 | 11 |
| Methionine (Met) | 2 | 1 |
| Phenylalanine (Phe) | 7 | 8 |
| Proline (Pro) | 7 | 7 |
| Serine (Ser) | 11 | 5 |
| Threonine (Thr) | 9 | 7 |
| Tryptophan (Trp) | 1 | 2 |
| Tyrosine (Tyr) | 3 | 3 |
| Valine (Val) | 13 | 18 |

Other amino acids known to be bonded to hemoglobin include the following: Nicotinic Acid, 2-Picolyliminomonoacetate, Benzyliminomonoacetate, 2,2′-bipyridine dicarboxylic acetate, 2-picolylimino diacetate, and Benzylimino diacetate.

Though not considered a part of the invention, the discussion that follows illustrates that syntheses of iron chelates of amino acids disclosed in the above table and those following the table are known in the art. Four amino acids have been selected solely for purposes of illustration.

2-picolyamine-One mole of 2-Picolylamine is dissolved in 200 cc of water. One mole of chloroacetic acid is slowly added together with one mole of sodium hydroxide solution resulting in the solution heating to an elevated temperature. The solution is then permitted to cool and then, ½ mole of ferrous sulfate is added while the temperature of the solution is maintained at 60° C. for 30 minutes. After cooling, ferrous 2-Picolyliminoacetate is formed and separated. In an alternative embodiment, to prepare the Benzyliminomonoacetate ferrous chelate, Benzylamine may be substituted for 2-Picolyamine.

Alanine-alanine is a ligand that has two donor groups that bond to iron. During the reaction, iron ions bond to the carboxyl group and the α-amino group of the alanine via the coordinate covalent bonds. The synthesis of bis alanine chelate is disclosed in numerous publications including Zargaran et al, Adv Pharm Bull. 2016 September; 6 (3): 407-413, Sep. 25, 2016 incorporated herein by reference. The process disclosed by Zargaran et al involves boiling 20 ml of deionized water for 5 minutes to remove dissolved oxygen. Then the temperature of the water is decreased to 70° C. and 3.47 grams of ferrous sulfate hepta hydrate (equivalent to 12.5 mmol) are added. The mixture is stirred constantly and the temperature maintained at about 70° C. until the iron salt completely dissolves. By decreasing the temperature of the mixture to 45° C., 4.27 grams of ascorbic acid (equivalent to 25 mmol) are added and stirred until the solution becomes clear. Then 2.25 grams of alanine (equivalent to 25 mmol) are added to the clear mixture with continuous mixing and the temperature is maintained at 45° C. Stirring follows for about further 4 hours. The prepared solution is dried using the lyophilization process and kept for further analysis.

Glycine—the formation of an iron chelate of glycine is disclosed by Hsinhung in U.S. Pat. No. 5,504,055A, granted Apr. 4, 1996 incorporated herein by reference. The process comprises bringing 1,000 grams of water to a boil and continuing to heat the water to boiling for 30 minutes to remove air. Thereafter, 133 grams of ferrous carbonate monohydrate are added to the boiling water. The mixture is stirred constantly and the temperature of the mixture maintained at about 80° C. Once the temperature reaches 80° C., 150 grams of glycine are then added to the ferrous carbonate solution. The temperature of the mixture is held at about 80° C. with continuous mixing until no additional material dissolves. The mixture is then filtered to remove the undissolved materials, the filtrate is dried at 110° C., and the dry material ground to produce a powder.

Arginine-formation of an iron chelate of arginine is disclosed by Ashmead et al in abandoned U.S. Patent application US20080194407A1 published Aug. 14, 2008 and incorporated herein by reference. The procedure disclosed for making the iron chelate of arginine involves adding 50 grams of citric acid to about 700 ml of deionized water and 616 grams of arginine to form a clear solution. To this solution of citric acid, arginine is slowly added together with 55.8 grams of elemental iron. The solution is heated to about 50° C. for 48 hours, or until substantially all the iron is observed to go into solution. The product can remain as a solution, or alternatively, cooled, filtered, and spray dried. The resulting product is a ferric trisarginate amino acid chelate.

To formulate an artificial blood composition, a single iron amino acid chelate may be used but desirably, mixtures of such chelates are used. The most preferred mixture would be the combination of all of the amino acid chelates shown in tables 1. If a mixture of chelates is used that is less than all of the chelates, it is desired that the mixture contain at least the following three amino acids known to be responsible for the most efficient bonding of oxygen to active sites-namely lysine, histidine, and aspartate.

Other than a mixture of all of the Table 1 amino acid chelates and those amino acid chelates further identified following Table 1 if desired, the following examples of suitable mixtures of iron amino acid chelates are shown in the following examples.

In hemoglobin, it is known that three key amino acid residues play a major role in the binding of oxygen to the active site: lysine, histidine, and aspartate. The following example represent desired mixtures of iron amino acid chelates for formulation of an artificial blood.

Example 1-A mixture of 35% by weight of the ferrous chelate of lysine, 25% by weight of ferrous chelate of histidine, 20% by weight of the ferric chelate of aspartate, 10% by weight of the ferrous chelate of glycine, and 10% by weight of the ferrous chelate of leucine.

Other suitable iron amino acid chelates are given in the following examples.

Example 2—A mixture of 35% by weight of the ferric chelate of lysine, 25% by weight of ferrous chelate of histidine, and 20% by weight of the ferric chelate aspartate.

Example 3—A mixture of 35% by weight of the ferrous chelate of lysine, 25% by weight of ferrous chelate of histidine, 20% by weight of the ferric chelate of alanine, 10% by weight of ferr[ic chelate of arginine, 5% by weight of the ferric chelate of glycine, and 5% by weight of the ferric chelate of leucine.

Example 4—A mixture of 25% by weight of the ferrous chelate of lysine, 25% by weight of ferric chelate of histidine, 20% by weight of the ferric chelate of aspartate, 10% by weight of the ferrous chelate of glycine, and 10% by weight of the ferric chelate of leucine, 10% by weight of the ferrous chelate of Tyrosine and a 5% by weight of the ferric chelate of Valine.

Other desirable combinations may be formulated using an Artificial Intelligence search platform.

To form an artificial blood composition from the mixtures of Examples 1 through 4, it is desirable to oxygenate the iron component and admix the chelates in a suitable solvent as will be discussed in greater detail below.

In addition to the above mixtures of iron amino acid chelates associated with hemoglobin, iron chelates of other amino acid chelates not typically associated with hemoglobin may be used in lesser amounts in the preparation of the artificial blood compositions described herein. Preferably, the concentration of those iron amino acid chelate not associated with hemoglobin would not exceed 25% by weight of the total artificial blood composition and more preferably, would not exceed 10% by weight of the artificial blood composition.

For purposes herein, a preferred iron amino acid chelate would be an iron chelate of pyridine or a bis-pyridine. Such chelates are known to the art and described in numerous publications and the preparation of these chelates is not considered to be a part of this invention.

The pyridine and bis pyridine starting materials may be represented by the following formulae:

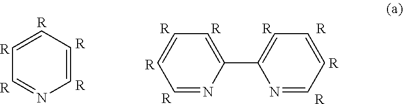

where at least one R is an alkyl amine. The alkyl amine may then be reacted with a sodium salt of methyl chloride which is then reacted with hydrochloric acid to form the pyridine carboxylic acid. As discussed above, the chelate is formed from a pyridine or bipyridine carboxylate—for example:

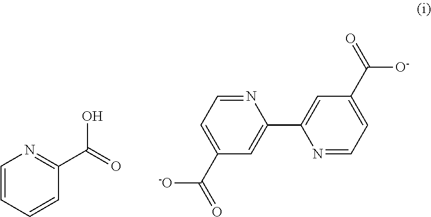

Methods for formation of metal chelates of pyridine and bipyridine caroxylates are disclosed in numerous publications inclusive of Methyl-Hydroxypyridinecarboxylic Acids as Possible Bidentate Chelating Agents for Aluminium (III): Synthesis and Metal-Ligand Solution Chemistry, VB Di Marco et al, European Journal of Inorganic Chemistry, VB Di Marco et al, 2002-Wiley Online Library, Volume 2002, Issue 10. Pages 1284-1293; Evaluation of 1-methyl-3, 4-hydroxypyridinecarboxylic acids as possible bidentate chelating agents for iron (III): Metal-ligand solution chemistry, Polyhedron 26 (13): 3227-3232, DOI: 10.1016/j.poly.2007.02.026, August 2007; Different approaches to the study of chelating agents for iron and aluminium overload pathologies, Guido Crispon et al, Anal Bioanal Chem. 2013 January; 405 (2-3): 585-601, doi: 10.1007/s00216-012-6468-7; 1, 6-Dimethyl-4-hydroxy-3-pyridinecarboxylic acid and 4-hydroxy-2-methyl-3-pyridinecarboxylic acid as new possible chelating agents for iron and aluminium, Dean et al, Dalton Trans. 2009 Mar. 14; (10): 1815-24. doi: 10.1039/b819148d. Epub 2009 Jan. 27. VB Di Marco et al, Inorganica, Chimica Acta, Volume 357, Issue 12, 10 Sep. 2004, Pages 3753-3758, and Neutral-Ligand Complexes of Bis(imino)pyridine Iron; and Synthesis, Structure, and Spectroscopy, SC Bart et al, norg. Chem. 2007, 46, 17, 7055-7063, Publication Date: Jul. 26, 2007, https://doi.org/10. 1021/ic700869h, each incorporated herein by reference for the teachings of amino acid complexes formed from pyridine based carboxylic acids.

Additional references relevant to the formation of such chelates include Neutral-ligand complexes of bis(imino)pyridine iron: Synthesis, structure, and spectroscopy, SC Bart, E Lobkovsky, E Bill, K Wieghardt, Chemistry, Inorg. Chem. 2007, 46, 17, 7055-7063, Publication Date: Jul. 26, 2007; Hydroxypyridinones with enhanced iron chelating properties. Synthesis, characterization and in vivo tests of 5-hydroxy-2-(hydroxymethyl)pyridine-4 (1H)-one, J. I. Lachowicz, V. M. Nurchi, G. Crisponi, M. G. Jaraquemada-Pelaez, M. Arca, A. Pintus, M. A. Santos b, C. Quintanova, L. Gano, Z. Szewczuk M. A. Zoroddu, M. Peana, A. Domínguez-Martin and D. Choquesillo-Lazarte, Dalton Trans., 2016, 45, 6517-6528; and Asymmetric diimine pyridine iron or cobalt complex catalyst; and Preparation Method and Application Thereof, CN102464677B granted Aug. 13, 2014, each incorporated herein for their teachings of metal-inclusive or iron-pyridine and bipyridine complexes.

Preferred iron amino acid chelates pyridine and bis pyridine include the following:

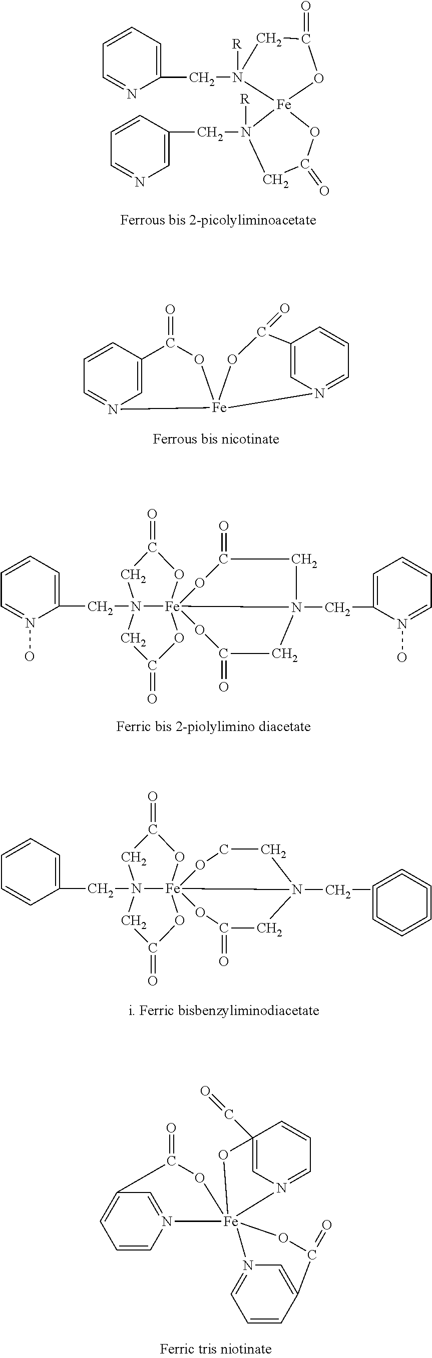

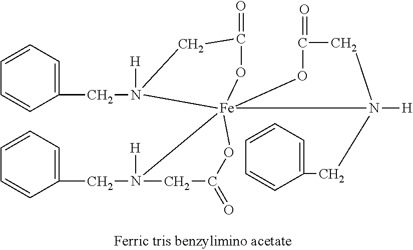

where, in the above formulae, R, where it appears is hydrogen, or an alkyl group having from 1 to 4 carbon atoms.

The following is an example of a mixture of chelates that may be utilized to form an artificial blood formulation containing iron chelates with different amino acids one of which is not related to hemoglobin.

Example 5—A mixture of 30% by weight of the ferrous chelate of lysine, 30% by weight of ferric chelate of histidine, 20% by weight of the ferric chelate of aspartate and 20% by weight of 5-amino pyridine-2-carboxylic acid.

It is known in the art that iron bonds reversibly to the iron atom of the heme group of hemoglobin. Each hemoglobin molecule can bind to four oxygen molecules. The reaction can be represented as: Hb+O₂≈HbO₂Hb+O₂≈HbO₂. The affinity of iron for oxygen refers to how strongly it binds to oxygen. The binding and release of oxygen by hemoglobin are influenced by various factors, including the partial pressure of oxygen, pH, temperature, and the presence of other molecules. Consequently, as is known to the art, hemoglobin may be oxygenated prior to use to enhance its oxygen carrying capability. This is a known procedure and is typically accomplished by passing a stream of oxygen gas through a solution of the hemoglobin or by contacting a solution of the hemoglobin with hydrogen peroxide.

The amino acid chelates comprising the artificial blood disclosed herein desirably are oxygenated using those known methods used to oxygenate hemoglobin—e.g, by passing a stream of oxygen through a solution of the amino acids or by contacting the amino acids with hydrogen peroxide. For example, for a 1 liter solution of the artificial blood of Example 1 above, the iron of the chelate may be oxygenated by passing from 1 to 6 liters of oxygen through the solution of the artificial blood for a period of about 2 to 10 minutes. Alternatively, oxygenation may take place by adding a solution of hydrogen peroxide to the solution of the artificial blood, for example, 10 ml of a 10% solution by weight of hydrogen peroxide may be added to 1 liter of an artificial blood solution formulated from the mixture of Example 1.

The majority of the iron amino acid chelates contemplated herein are viscous liquids or solids and would require a solvent for the chelate mixture to enable transfusion of the artificial blood into an individual. When a solvent or diluent is used, the composition desirably utilizes a pharmaceutically acceptable carrier, such as a diluent fraction comprising a salt. The salt can be essentially any salt, though the salts presently preferred are those that are pharmaceutically acceptable for delivery to mammals, e.g., a saline solution of sodium chloride. The compositions of the invention are isotonic, hypertonic or hypotonic. In various embodiments, the composition is hypertonic. In an exemplary embodiment, the composition included sufficient sodium chloride to render it hypertonic. In other embodiments, the diluent is tonic phosphate buffered saline.

For use of a chelated amino acid as an oxygen carrier blood replacement formulation, pharmaceutical grade amino acid chelates, free of interfering anions are needed. A method for preparing a pharmaceutically acceptable amino acid chelate is disclosed by Ashmead, U.S. Pat. No. 4,830,716A, granted May 16, 1989 and incorporated herein by reference.

Additional methods for forming pharmaceutically acceptable amino acid chelates suitable for use in accordance with the subject invention include: Asmead, CA1299812C granted Apr. 29, 1992; Ashmead, EP0256645A2 published Dec. 11, 1991; CN100540031C published Sep. 16, 2009; and Park, US20100093850A1 published Apr. 15, 2010.

In another aspect of the invention, the artificial blood formulation also comprises enzymes, preferably in an amount of from about 0.1 to about 0.3 percent by weight. Enzymes play an important role in oxygen transport within cells primarily through their involvement in cellular respiration, the process by which cells generate energy in the form of ATP (adenosine triphosphate) through the oxidation of glucose. Enzymes are not directly involved in oxygen transport in the same way as proteins such as hemoglobin, which bind and carry oxygen in the bloodstream. However, enzymes play a significant role in processes that support oxygen transport and utilization within the body. The enzymes assist in oxygen transport within cells primarily through their involvement in cellular respiration, the process by which cells generate energy in the form of ATP (adenosine triphosphate) through the oxidation of glucose.

The key steps in oxygen transport within cells include:
Glycolysis: In the cytoplasm, the enzyme hexokinase catalyzes the first step of glycolysis, where glucose is phosphorylated to glucose-6-phosphate. Later, phosphofructokinase catalyzes the conversion of fructose-6-phosphate to fructose-1,6-bisphosphate, an important regulatory step in glycolysis.
Krebs Cycle (Citric Acid Cycle): In the mitochondria, several enzymes are involved in the Krebs cycle, which is the central metabolic pathway for the oxidation of acetyl-CoA derived from glucose, fatty acids, and amino acids. Enzymes such as citrate synthase, isocitrate dehydrogenase, α-ketoglutarate dehydrogenase, and succinate dehydrogenase are key in this cycle, facilitating the conversion of various substrates and generating reducing equivalents like NADH and FADH2.
Electron Transport Chain (ETC): Within the inner mitochondrial membrane, the ETC consists of a series of protein complexes (I, II, III, and IV) and coenzymes (e.g., ubiquinone, cytochrome c) that transfer electrons from NADH and FADH2 to oxygen, ultimately generating a proton gradient across the membrane. Enzymes within the complexes catalyze these electron transfer reactions.
Oxygen as Final Electron Acceptor: At the end of the ETC, oxygen serves as the final electron acceptor, combining with protons to form water. The enzyme cytochrome c oxidase (Complex IV) catalyzes the transfer of electrons from cytochrome c to oxygen, facilitating this reaction.

Examples of key enzymes involved in oxygen transport indirectly include:
Cytochrome c Oxidase: This enzyme is found in the mitochondria and is responsible for the final step in the electron transport chain during cellular respiration. It transfers electrons to molecular oxygen, which is the final electron acceptor, resulting in the production of water. This process is essential for generating ATP, the energy currency of cells, which is needed for various cellular activities, including muscle contraction and active transport.
Superoxide Dismutase (SOD): SOD is an antioxidant enzyme that catalyzes the dismutation of the superoxide radical (O2−) into oxygen and hydrogen peroxide. By neutralizing superoxide radicals, SOD helps prevent oxidative damage to cells and tissues, which can impair oxygen transport and utilization.
Carbonic Anhydrase: While not directly involved in oxygen transport, carbonic anhydrase facilitates the conversion of carbon dioxide (CO2) to bicarbonate ions (HCO3−) in red blood cells. This reaction is crucial for the transportation of CO2 from tissues to the lungs for elimination via respiration, which indirectly supports efficient oxygen transport by maintaining appropriate pH levels in the blood.
Catalase: Similar to SOD, catalase is an antioxidant enzyme that catalyzes the decomposition of hydrogen peroxide into water and oxygen. By removing hydrogen peroxide, which can generate reactive oxygen species (ROS), catalase helps protect cells from oxidative damage and supports overall cellular health, indirectly aiding in oxygen transport and utilization.

The enzymes may be human enzymes, animal enzymes, or mixtures of the two.

Preferably, the enzymes would be added to the formulation during the step of oxidation of the artificial blood as described above. The amino acid chelates of the present invention may be administered into the circulatory system by injecting the composition directly and/or indirectly into the circulatory system of the subject, by one or more injection methods. The preferred method is by intravascular injections, such as intravenous and intra-arterial injections. Accordingly, to prepare an artificial oxygen carrier blood composition using the amino acid chelates, the chelate would be dissolved in a pharmaceutically acceptable carrier as needed. An intravenous solution would be an acceptable carrier. Of the various intravenous solutions, isotonic saline (0.9%) is recommended for use with the chelate. Other commonly used intravenous solutions might cause varying degrees of difficulty when mixed with the chelates. The concentration of the chelate in the saline solution may vary from about 5 percent by weight up to saturation but preferably varies between 20 percent by weight up to 80 percent and most preferably varies between 40 percent by weight up to 75 percent by weight.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A process for treating an individual suffering trauma from loss of blood with an artificial blood composition, said process comprising the step of infusing said individual with a pharmaceutically acceptable saline solution of a hemoglobin free oxygen carrying mixture or solution of one or more iron amino acid chelates where the amino acid used to form the chelate is selected from those amino acids otherwise known to be bonded to hemoglobin.

2. The process of claim 1, wherein the amino acid used to form the chelate is one or more amino acids selected from the group consisting of Alanine, Arginine, Asparagine, Aspartic Acid, Cysteine, Glutamine, Glutamic Acid, Glycine, Histidine, Isoleucine, Leucine, Lysine, Methionine, Phenylalanine, Proline, Serine, Threonine, Tryptophan, Tyrosine, Valine, and mixtures thereof.

3. The process of claim 1, wherein the amino acid used to form the chelate is a mixture of Alanine, Arginine, Asparagine, Aspartic Acid, Cysteine, Glutamine, Glutamic Acid, Glycine, Histidine, Isoleucine, Leucine, Lysine, Methionine, Phenylalanine, Proline, Serine, Threonine, Tryptophan, Tyrosine, and Valine.

4. The process of claim 2, wherein the formulation contains at least one additional iron amino acid selected from the group consisting of Nicotinic Acid, Picolyliminomonoacetate, Benzyliminomonoacetate, 2,2′-bipyridine dicarboxylate, 2-picolylimino diacetate, and Benzylimino diacetate in an amount not exceeding 25% by weight of the total of the amino acid chelates.

5. The process of claim 2, wherein the amino acid used to form the chelate includes at least one of a member selected from the group consisting of lysine, histidine, and aspartate.

6. The process of claim 5, wherein the amino acid used to form the chelate is a mixture of amino acids consisting of lysine, histidine, and aspartate.

7. The process of claim 5, wherein the amino acid used to form the chelate is a mixture of 35% by weight of the ferrous chelate of lysine, 25% by weight of ferrous chelate of histidine, 20% by weight of the ferric chelate of aspartate, 10% by weight of the ferrous chelate of glycine, and 10% by weight of the ferrous chelate of leucine.

8. The process of claim 2, wherein the amino acid solution further contains an iron amino acid chelate that is formed from an amino acid other than amino acid known to be bonded to hemoglobin.

9. The process of claim 8, wherein the iron amino acid chelate not bonded to hemoglobin is a chelate of a member selected from the group of pyridine and bipyridine.

10. The process of claim 2, wherein the iron component of the amino acid has been oxygenated by passing oxygen through a solution of the amino acids.

11. The process of claim 2, wherein the iron component of the amino acid has been oxygenated by the addition of hydrogen peroxide to a solution of the amino acid chelates.

12. The process of claim 2, wherein the iron amino acid chelate is in a saline solution where the concentration of the iron amino acid chelate in solution varies between 5 percent by weight to saturation.

13. The process of claim 12, wherein the concentration of the iron amino acid chelate in solution varies between 40 percent by weight up to 75 percent by weight.

14. The process of claim 1, wherein the hemoglobin free mixture or solution also comprises one or more human or animal enzymes.

15. The process of claim 14, wherein the one or more enzymes are selected from the group consisting of hexokinase, phosphorfructokinase, citrate synthase, isocitrate dehydrogenase, α-ketoglutarate dehydrogenase, succinate dehydrogenase, ubiquinone, cytochrome c, cytochrome c oxidase, superoxide dismutase, carbonic anhydrase, catalase, and mixtures of two more thereof.

16. An artificial hemoglobin free artificial blood composition, said composition comprising a pharmaceutically acceptable saline solution of a hemoglobin free mixture or solution comprising one or more iron amino acid chelates where the amino acid used to form the chelate is selected from those amino acids otherwise known to be bonded to hemoglobin.

17. The composition of claim 16, wherein the amino acid used to form the chelate is one or more amino acids selected from the group consisting of Alanine, Arginine, Asparagine, Aspartic Acid, Cysteine, Glutamine, Glutamic Acid, Glycine, Histidine, Isoleucine, Leucine, Lysine, Methionine, Phenylalanine, Proline, Serine, Threonine, Tryptophan, Tyrosine, Valine, and mixtures thereof.

18. The composition of claim 16, wherein the amino acid used to form the chelate is a mixture of Alanine, Arginine, Asparagine, Aspartic Acid, Cysteine, Glutamine, Glutamic Acid, Glycine, Histidine, Isoleucine, Leucine, Lysine, Methionine, Phenylalanine, Proline, Serine, Threonine, Tryptophan, Tyrosine, and Valine.

19. The composition of claim 17 which comprises at least one additional iron amino acid selected from the group consisting of Nicotinic Acid, Picolyliminomonoacetate, Benzyliminomonoacetate, 2,2′-bipyridine dicarboxylate, 2-picolylimino diacetate, and Benzylimino diacetate in an amount not exceeding 25% by weight of the total of the amino acid chelates.

20. The composition of claim 16, wherein the amino acid used to form the chelate includes at least one of a member selected from the group consisting of lysine, histidine, and aspartate.

21. The composition of claim 16, wherein the amino acid used to form the chelate is a mixture of amino acids consisting of lysine, histidine, and aspartate.

22. The composition of claim 16, wherein the iron component of the amino acid has been oxygenated by passing oxygen through a solution of the amino acids.

23. The process of claim 16, wherein the hemoglobin free mixture or solution also comprises one or more human or animal enzymes.

24. The process of claim 23, wherein the one or more enzymes are selected from the group consisting of hexokinase, phosphorfructokinase, citrate synthase, isocitrate dehydrogenase, α-ketoglutarate dehydrogenase, succinate dehydrogenase, ubiquinone, cytochrome c, cytochrome c oxidase, superoxide dismutase, carbonic anhydrase, catalase, and mixtures of two more thereof.
